(19) United States
(12) Patent Application Publication
(10) Pub. No.: US 2002/0188207 A1
(43) Pub. Date: Dec. 12, 2002

(54) Anchor for sensor implanted in a bodily lumen

(76) Inventor: Jacob Richter, Ramat Hasharon (IL)

Correspondence Address: MORGAN & FINNEGAN, L.L.P., 345 Park Avenue, New York, NY, 10154-0053, US

(21) Appl. No.: 10/139,113

(22) Filed: May 2, 2002

Related U.S. Application Data
(63) Continuation-in-part of application No. 09/004,420, filed on Jan. 8, 1998.

(51) Int. Cl. A61B 5/02

(52) U.S. Cl. 600/486

(57) ABSTRACT

A device and method for centrally positioning a sensor in a bodily lumen. A sensor support is coupled to an anchor that is inserted into a bodily lumen. The anchor is then secured within the bodily lumen, in one embodiment, by attaching the support legs of the anchor to the bodily lumen. The support legs of the anchor have protrusions to attach to the walls of the bodily lumen. The sensor is positioned at the center of the lumen to more accurately measure the physiological properties and biochemical parameters of the lumen. The anchor may be inserted during an intervention procedure or during a special insertion procedure. The sensor may be remotely interrogated exterior to the bodily lumen periodically or continuously. Furthermore, the sensor may be provided with a protective coating to protect it from damage during insertion.

### CROSS-REFERENCE TO RELATED APPLICATIONS

This application is a continuation in part of co-pending U.S. patent application Ser. No. 09/004,420 entitled Method and Device For Fixation of a Sensor in a Bodily Lumen, filed in the name of Richter et al. on Jan 8, 1998, the entirety of which is incorporated by reference herein.

### FIELD OF THE INVENTION

The present invention relates generally to an anchor and method for attaching a sensor in a bodily lumen, more specifically, the anchor provides for fixation of the sensor at the center of a bodily lumen, such as a coronary artery or other vessels, for taking more accurate measurements within the lumen and generally where the position at the center of the lumen is more desirable.

### BACKGROUND OF THE INVENTION

The circulatory system is a complex network of arteries and veins that connects the heart to every area of the body. Physicians have been able to utilize long flexible catheters to deliver specialized tools and medicine to diagnose and treat heart disease. The entrance to the arterial system is typically through a needle puncture made in the femoral artery. The brachial and radial arteries can also be utilized.

Sensors for the monitoring and/or recording of various human physical, chemical and/or physiological parameters are well known. U.S. Pat. No. 4,485,813 describes a sensor that may be permanently implanted in a specific location within the human body in an implantable medical device such as a pacemaker. This sensor is used to monitor certain physical and/or physiological parameters of the subject in which it has been implanted. This sensor can be maintained in the subject for extended periods of time to continuously monitor information about the subject using the pacemaker.

A severe limitation to the sensor described in U.S. Pat. No. 4,485,813 is the limited number of possible locations in which it can be implanted due to the requirement that the sensor must be located in a medical device. In addition, it is difficult to independently affix the sensor. These limitations, location and fixation of the sensor, limits the usefulness of the sensor for inter-lumen applications.

Sensors used to monitor parameters within lumens may be highly sensitive to mechanical pressure. As a result there is a great risk of the sensor being damaged during insertion and or positioning. Damage to the sensor could result in poor performance or non-operability of the sensor. For example, should some parts of a sensor break during insertion, the sensor would be rendered inoperable. Due to the risks associated with the procedures for the insertion of sensors, there would be great costs and risks involved should a sensor be damaged or destroyed during insertion.

Physiological conditions and biochemical parameters within a lumen can differ when measured at different distances from the walls of the lumen, as fluid flow at the wall of a bodily lumen may not accurately represent fluid flow through the bodily lumen overall. Additionally, disposing a sensor adjacent to the wall of a lumen may cause the sensor to be covered with tissue from the lumen wall, thereby causing a negative effect on the functionality of the sensor if its exposure to the fluid is vital to its operation.

Thus, a need exists for a device and method of disposing a sensor such that an accurate measurement of physiological conditions and biological parameters within the lumen can be taken. In addition, there is also a need for a device and method of protecting sensors during insertion and fixation.

### SUMMARY OF THE INVENTION

The present method and device avoids many of the disadvantages of previous attempts to circumvent these problems. In one embodiment, an anchor is delivered with a sensor through a catheter and inserted in a bodily lumen. The sensor is positioned about in the center of the lumen with minimal disturbance to blood flow in the lumen. The anchor may be made of a super-elastic wire-like material and may have support legs with protrusions or hooks or the like. When the anchor is deployed from the catheter legs of the anchor expand or spring outwardly with these protrusions to attach the lumen wall. The sensor is then fixated at the center of the lumen with minimal invasive sensor deployment and minimal disturbance to flow. The sensor may monitor various properties concerning the blood vessel, the fluid and the flow in it, to allow physicians to assess the condition of a patient.

Depending on the embodiment, multiple sensors may be deployed on the same anchor allowing for positioning of sensors at a prescribed distance from each other. In addition, depending on the embodiment, the sensor may be mounted using a single support leg. The support leg may have one or many protrusions that allow the support leg to be mounted to the lumen wall. These, and other aspects of the anchor device and method of insertion, are described in the following brief and detailed description of the drawings.

### BRIEF DESCRIPTIONS OF THE DRAWINGS

Further aspects of the instant invention will be more readily appreciated upon review of the detailed description of the preferred embodiments included below when taken in conjunction with the accompanying drawings, of which:

FIG. 1A is a drawing of a first fixation device for a sensor according to a preferred embodiment of the present invention;

FIG. 1B is an illustration of a method for fixation of the sensor of FIG. 1A within a lumen according to a preferred embodiment of the present invention;

FIGS. 2A and 2B, show illustrations of a second fixation device for a sensor before expansion and after expansion, respectively, according to a preferred embodiment of the present invention.

FIGS. 3A and 3B, show illustrations of a third fixation device for a sensor before expansion and after expansion, respectively, according to a preferred embodiment of the present invention.

FIG. 4, illustrates a second method for fixation of a sensor within a lumen using the third fixation device shown in FIGS. 3A and 3B, according to a preferred embodiment of the present invention.

FIG. 5, shows an illustration of a mask for etching of a flat design of the fixation device of FIGS. 3A and 3B, according to a preferred embodiment of the present invention.

FIG. 6, shows an illustration of a mask for etching of a flat design of the fixation device of FIGS. 2A and 2B, according to a preferred embodiment of the present invention.

FIG. 7, shows a fourth fixation device for a sensor before expansion, according to a preferred embodiment of the present invention.

FIG. 8, shows an illustration of a mask for etching of a flat design of the fixation device of FIG. 7, according to a preferred embodiment of the present invention.

FIGS. 9A and 9B, show an enlarged side view of a cross section of the sensor support from FIG. 3A along the line formed between points a′ and a′ according to two different embodiments of the present invention.

FIG. 10A shows an exemplary anchor for a sensor to be positioned at the center of a bodily lumen.

FIG. 10B shows a catheter for delivering the anchor to a bodily lumen.

FIG. 10C shows an anchor attached to a bodily lumen, with the sensor positioned at the center of the bodily lumen.

FIG. 10D shows another exemplary anchor for a sensor to be positioned at the center of a bodily lumen.

FIG. 10E shows exemplary support legs of the anchor shown in FIG. 10D.

### DETAILED DESCRIPTION OF THE INVENTION

Described herein is a method and device for fixation of a sensor in a bodily lumen. Through the use of such a method and device, remotely interrogated sensors may be fixed within bodily lumens. Such sensors may be used to record and/or monitor parameters such as, for example, physiological parameters, e.g., pressure and velocity of flow, and biochemical parameters, e.g., level of gases and biochemical substances in the fluid contained in the lumen.

The monitoring of conditions in lumens today dictates some level of intervention and the frequency of such monitoring may be limited by the relative risk of the required intervention. The present invention, therefore, allows for a sensor device, which may be implanted, either temporarily or permanently, in a lumen and interrogated from an exterior position, for example, the surface of the body, at any time without any intervention or physical intrusion.

The present invention provides a method and device for the fixation of such sensors in specific desired locations and/or preferred positions in the lumen. Such fixation of the sensors may be achieved at the time of any required surgical or minimally invasive intervention or independently by catheterization. Furthermore, the sensor may be connected to the repair device, e.g., the stitches of a bypass, an aneurismal repair device, a stent, etc., or mounted on its own dedicated fixation device.

A sensor may be fixed inside a lumen by any number of means, including directly attaching the sensor in place, for example, by including holes in the sensor, e.g., around its periphery, and attaching the sensor to the stitches of a bypass during surgery, or through the use of a surgical adhesive. A sensor may also be positioned inside a lumen using a carrier or support (of any shape and size) which may be part of, or coupled to a repair device, e.g., a stent or aneurismal correction device which holds the sensor in place adjacent to or near the repair device. Additionally, a sensor may be positioned inside a lumen using a dedicated device, e.g., an anchoring ring, which is held within a lumen and fixed in place, for example, by expansion with a catheter balloon. The anchoring ring does not necessarily have to be circular in shape, but may instead be oval or any other shape best suited for the location where placed. Additionally, the anchoring ring may have a separate carrier or support to hold the sensor. The carrier or support may be any shape or size, including, for example, circular, square, rectangular, diamond shaped, linear with or without a bent or curved end, etc, and it may be constructed as only a border or as a solid piece of material.

Multiple sensors may be attached to a carrier or carriers, for example, two sensors with one placed on each side of a stent, or two sensors attached at both connections of a bypass section, e.g., one sensor at the entrance to an aneurismal sleeve and one at the outside of the sleeve to monitor for a possible leak around the sleeve. Additionally, a sensor may have multiple repair devices or dedicated devices supporting it within a lumen, either with or without a carrier, for example, a sensor supported between two anchoring rings.

A sensor may be supported by or connected to a carrier, for example, by providing a groove-like depression(s) or notch-like depression(s) in the sensor into which a portion(s) of the carrier may be inserted, or the sensor may be configured such that a portion(s) of the sensor, for example, a lip-like extension(s) or protrusion(s), may extend beyond the dimensions of the carrier to be supported thereby. Additionally, the sensor may be attached to the carrier, for example, by welding and/or bonding by an adhesive or any combinations of the above.

After a sensor is fixed within a lumen, for example, during an intervention procedure such as aneurismal device implantation, PTCA, coronary bypass surgery, etc., it may thereafter be monitored periodically to track any of a variety of parameters or to assess the effectiveness of the procedure that was performed. For example, the sensor may be monitored periodically to assess the long term progress or deterioration of the corrective effect, and the progress of relevant symptoms of a disease.

Multiple sensors may be implanted and may be monitored individually or simultaneously to derive gradients along a lumen and across a repair device or section. Such sensors may be fixed in any number of positions within a lumen, for example, on both sides of a lesion treated by PTCA with or without a stent, on both sides of a bypass section, and before, after and around an aneurismal repair device.

The device also provides for a device and method for the protection of sensors during insertion. In order to preserve sensors during insertion and remove the risk of damage to or destruction of sensors during an insertion or positioning procedure, sensors may be coated with a protective layer which is soluble in an aqueous solution, and which disappears immediately or soon after deployment of the sensor in the body. The material used for, thickness of, and hardness of the coating may vary, for example, depending on the location of the sensor, the type of sensor, protection level sought, and rate of dissolution desired.

The fixation device may be constructed by first creating a flat version of the desired pattern for the fixation device, for example, from a piece of thin stainless steel sheet metal or some other material, e.g., any metal, non-metallic or bioabsorbable material. The flat pattern can be produced through any suitable technique, such as etching the design into the sheet metal, by cutting with a very fine laser, or by any other technique.

Once the material has been cut, it is deformed so as to cause its edges to meet. To create a fixation device from a flat, metal pattern, the flat metal is rolled until the edges meet. The portion which holds the sensor may be located along the circumference of the fixation device, may extend perpendicular to the cross-section of the ring formed or may extend in some other manner from the ring formed by the fixation device. The locations where edges meet are joined together, such as by spot welding. Afterwards, the fixation device is polished, either mechanically or electrochemically.

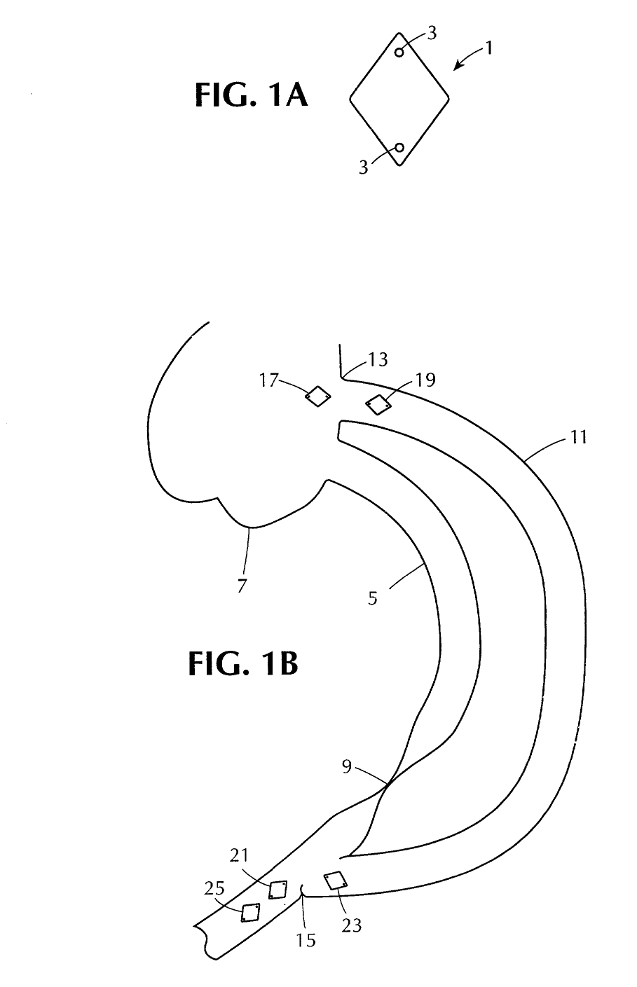

Reference is now made to FIGS. 1A and 1B, which illustrate a first fixation device for a sensor and a first method for fixation of a sensor within a lumen, respectively, according to a preferred embodiment of the present invention.

In FIG. 1A, there is shown a sensor 1 having two holes 3 in its periphery for attachment to sutures within a lumen. In FIG. 1B, there is shown a coronary artery 5 starting at the Aorta 7 and having an occlusion 9. A bypass 11 is connected between the Aorta at point 13 and at point 15 beyond the occlusion 9. Sensor 1 is placed either at the proximal ostium 17 or at the proximal part of the bypass 19. Alternatively, sensor 1 may be placed at the distal ostium 21, at the distal part before the distal ostium 23, or at the distal part after the distal ostium 25. Any number of sensors may be used, and they may be placed in any combination of the above positions or any other position desired. The sensor 1 is fixed in place using the two holes 3 for attachment to the sutures. Alternatively, the sensor 1 may be fixed in place using surgical adhesive or a surgical staple(s).

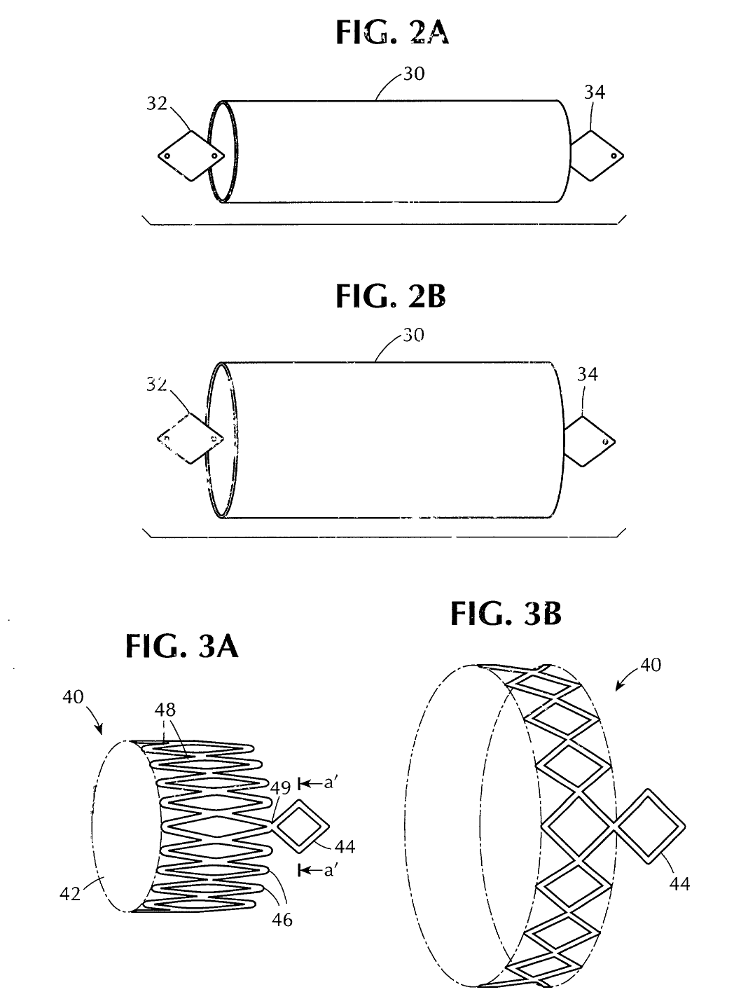

Referring now to FIGS. 2A and 2B, there are shown a second fixation device for a sensor before expansion and after expansion, respectively, according to a preferred embodiment of the present invention.

In FIG. 2A, there is shown a stent 30 in a non-expanded state with a first sensor support 32 and a second sensor support 34. Alternatively, the stent 30 may include more than two sensor supports. For example, a third sensor support may be located opposite the first sensor support 32. In FIG. 2B, the stent 30 from FIG. 2A is shown in its expanded state. Expansion may be accomplished, for example, by balloon catheterization or some other procedure. To fix a sensor within a lumen, the stent 30 is positioned as it normally is during any medical procedure in which a stent is used. Prior to expansion, and either prior to or after insertion of the stent 30 into the lumen, a sensor is placed in, placed on or attached to the first sensor support 32 and/or the second sensor support 34. The stent 30 is then either expanded, or inserted into the lumen and then expanded. The same procedure may be used to fix any number of sensors within a lumen, with the additional step of placing each sensor either in or on, or attaching each sensor to its corresponding sensor support.

Referring now to FIGS. 3A and 3B, there are shown a third fixation device for a sensor before expansion and after expansion, respectively, according to a preferred embodiment of the present invention.

In FIG. 3A, there is shown a fixation device 40 in the form of an anchoring ring 42, in a non-expanded state coupled to a sensor support 44. The fixation device 40 may be formed of any malleable material, which does not revert automatically to its original shape after being expanded. The anchoring ring 42 is made up of a plurality of elliptical sections 46 connected one to the other at the middle of each of their long portions 48 to form a ring. The sensor support 44 is connected to one of the elliptical sections 46 at a short portion 49, and perpendicular to a cross-section of the anchoring ring 42 forming a circular plane. The sensor support 44 is formed in the shape of a diamond, but can be any shape desired. Additionally, there may be multiple sensor supports attached to the anchoring ring 42. Alternatively, the anchoring ring 42 may be made of a single sinusoidal ring, with one or more sensor supports attached to the peaks, since it does not serve any support function for the lumen.

FIG. 3B shows the fixation device 40 of FIG. 3A in an expanded state. To fix a sensor within a lumen, the fixation device 40 is positioned within the lumen, for example, during an intervention procedure, and expanded, for example, by balloon catheterization or some other procedure. Prior to expansion, and either prior to or after insertion of the fixation device 40 into the lumen, the sensor is placed in, placed on or attached to the sensor support 42. The fixation device 40 is then either expanded, or inserted into the lumen and then expanded. The same procedure may be used to fix multiple sensors within a lumen, with the additional step of placing each sensor either in or on, or attaching each sensor to a corresponding sensor support.

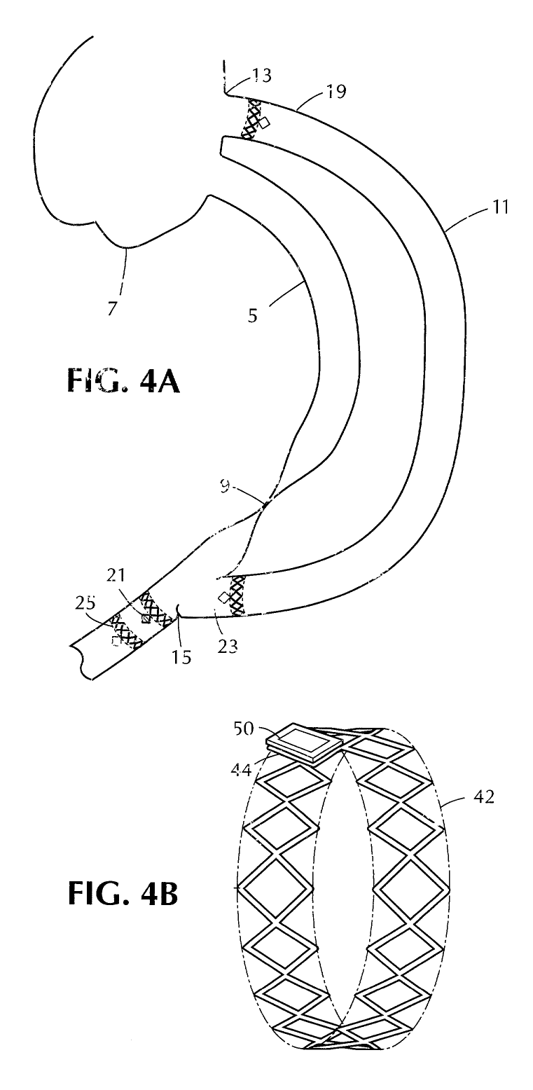

Referring now to FIG. 4, which illustrates a second method for fixation of a sensor within a lumen using the third fixation device shown in FIGS. 3A and 3B, according to a preferred embodiment of the present invention. As illustrated in FIG. 1B, a coronary artery 5 starting at the Aorta 7 and having an occlusion 9 is fitted with a bypass 11, which is connected between the Aorta at point 13 and at point 15 beyond the occlusion 9. Sensor 50, which is carried by the sensor support 44 coupled to the anchoring ring 42 of FIGS. 3A and 3B, is placed either at the proximal part of the bypass 19, at the distal ostium 21, at the distal part before the distal ostium 23, or at the distal part after the distal ostium 25. Any number of sensors may be used, and they may be placed in any combination of the above positions or any other position desired in which an anchoring ring can be used. The sensor 50 is fixed in place by expansion using balloon catheterization.

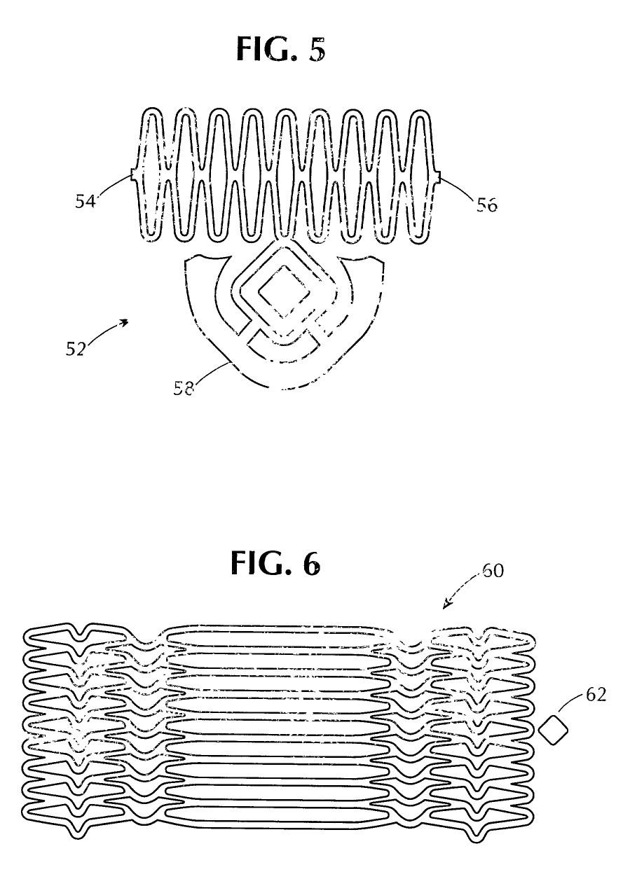

Referring now to FIG. 5, there is shown an illustration of a mask for etching of a flat design of the fixation device of FIGS. 3A and 3B, according to a preferred embodiment of the present invention. A mask 52 is created for etching a flat design of a fixation device. The flat design is then etched onto a piece of thin sheet metal or some other malleable material. The flat design is next cut from the sheet metal using, for example, a fine laser. The cut flat design is then polished and bent into a circular (or other) shape. Points 54 and 56 show the locations where the flat design is coupled, for example, by welding after it is bent. The welding creates an anchoring ring. Sensor support 58 is positioned approximately at the midpoint of the mask 52, but may alternatively be located at any other position. Additionally, there may be multiple sensor supports, for example, located at both sides of the fixation device design.

Referring now to FIG. 6, there is shown an illustration of a mask for etching of a flat design of the fixation device of FIGS. 2A and 2B, according to a preferred embodiment of the present invention. A mask 60 is created for etching a flat design of a stent. The flat design is then etched onto a piece of thin sheet metal or some other malleable material. The flat design is next cut from the sheet metal using, for example, a fine laser. The cut flat design is then polished and bent into a circular (or other) shape and coupled, for example, by welding after it is bent. Sensor support 62 is positioned approximately at the midpoint of the mask 60, but may alternatively be located at any other position. Additionally, there may be multiple sensor supports, for example, located at both sides of the stent design.

Referring now to FIG. 7, there is shown a fourth fixation device for a sensor before expansion, according to a preferred embodiment of the present invention. The fixation device 70 in the form of a dual anchoring ring comprises a first ring 72 and a second ring 74, in a non-expanded state, with a sensor support 76 positioned between the two rings 72, 74. The fixation device 70 may be formed of any malleable material, which does not revert automatically to its original shape after being expanded. The fixation device 70 is made up of a plurality of sections 78 connected one to the other to form two anchoring rings 72, 74. A sensor support 76 is connected to one of the sections 78 of each anchoring ring 72, 74 perpendicular to a cross-section of each of the rings 72, 74 forming a circular plane, and is positioned between the two rings 72, 74. The sensor support is formed in the shape of a diamond, but can be any shape desired. Additionally, there may be multiple sensor supports attached to the fixation device 70. Alternatively, the fixation device 70 may be made of two single sinusoidal rings, with one or more sensor supports attached to the peaks, since it does not serve any support function for the lumen. The fixation device 70 may alternatively be made of two stents, one on each side of a sensor support, or having multiple sensor supports attached thereto.

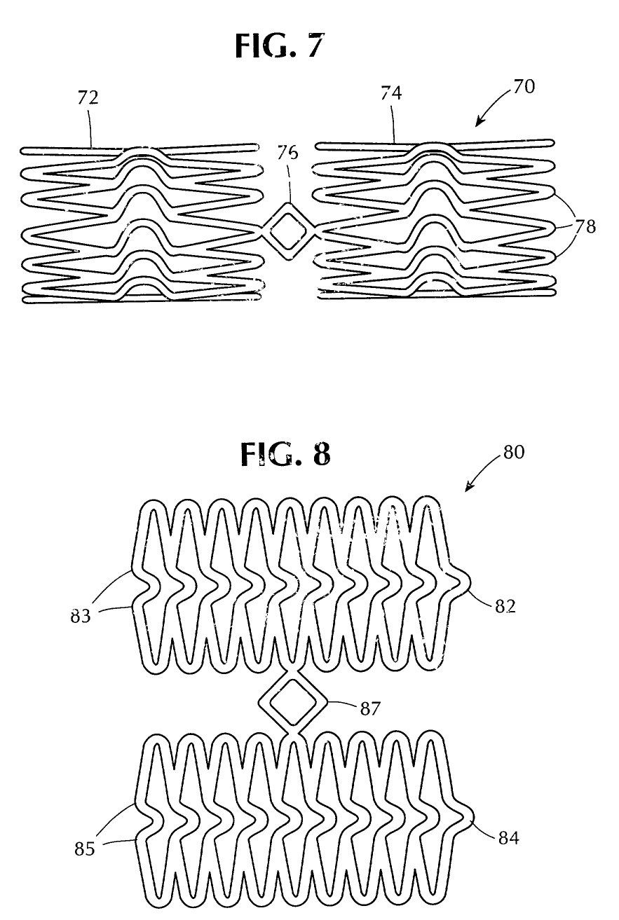

Referring now to FIG. 8, there is shown an illustration of a mask for etching of a flat design of the fixation device of FIG. 7, according to a preferred embodiment of the present invention. A mask 80 is created for etching a flat design of the fixation device. The flat design is then etched onto a piece of thin sheet metal or some other malleable material. The flat design is next cut from the sheet metal using, for example, a fine laser. The cut flat design is then polished and bent into a circular (or other) shape. Points 82 and 83, and points 84 and 85 show the respective locations where the flat design is coupled, for example, by welding after it is bent. The welding creates two anchoring rings. Sensor support 87 is positioned approximately at the midpoint of the mask 80, but may alternatively be located at any other position. Additionally, there may be multiple sensor supports, for example, located at both sides of the fixation device design.

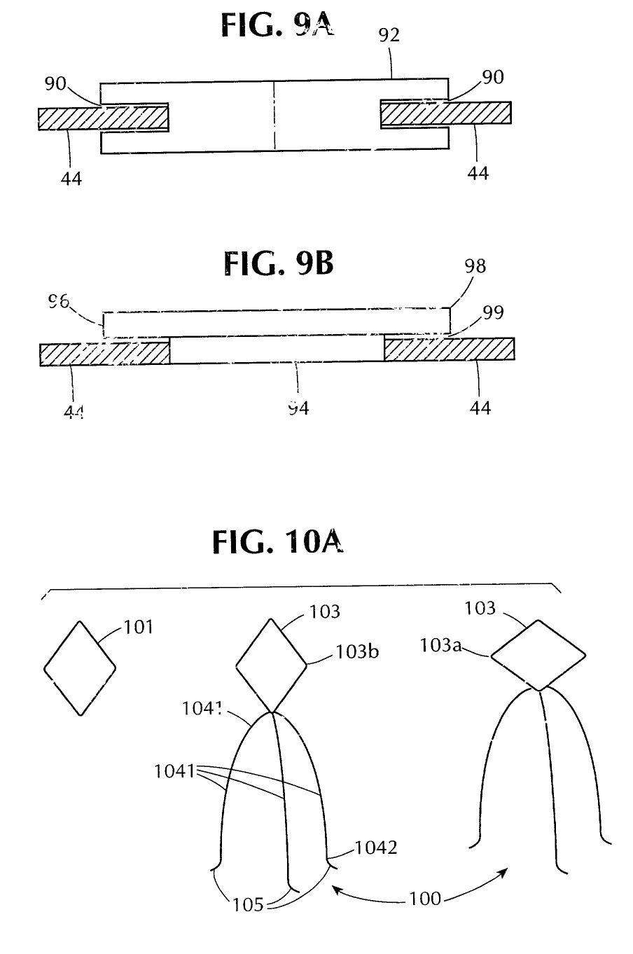

Referring now to FIGS. 9A and 9B, there is shown an enlarged side view of a cross section of the sensor support from FIG. 3A along the line formed between points a′ and a′. As shown in FIG. 9A, a groove 90 is formed in two portions of the periphery of sensor 92, for example, by cutting with a wire saw, by etching, by laser cutting, etc., and the sensor 92 is then inserted into the sensor support 44 such that two portions of the sensor support 44 are positioned within the groove 90 providing support for the sensor 92. Alternatively, instead of the grooves, two notches may be formed in the periphery of the sensor 92 in which the two portions of the sensor support 44 may be positioned.

As shown in FIG. 9B, sensor 94 is formed with a lip 96 around its upper edge 98. Sensor 94 may instead be formed with one or more protrusions along its upper edge 98. Alternatively, the lip or protrusion(s) may be located on the bottom or at any other position on the sensor. The sensor 94 is coupled to the sensor support 44, for example, by bonding by an adhesive, welding, soldering, etc., the lip 96 or protrusion(s) to an edge or portion 99 of the sensor support. Alternatively, the sensor 94 may be placed on the sensor support 44 and supported by the lip 96 or by the protrusion(s).

Due to the sensitivity of the sensors that are used for monitoring, which have very thin membranes that are extremely sensitive to mechanical pressure, a coating may be placed on the sensors to protect them from damage and/or destruction during deployment. The coating may be made from a material that is soluble in an aqueous solution, and may dissolve immediately or soon after deployment of the sensor. The material used, the thickness of the coating and the hardness of the coating will depend to a large extent on the location of the sensor, the type of sensor, and a variety of other factors including the physiology involved, the parameters being measured, and the desired speed of deployment.

A first example of a coating is a composition comprising solidified sugar syrup made of approximately equal amounts of glucose and sucrose. The proportions of glucose and sucrose may be varied, however, depending on the application.

A second example of a coating is a composition comprising Hydroxy Propyl Methyl Cellulose, Hydroxy Propyl Cellulose and Colloidal Silicone Dioxide, all finely ground and mixed in water, which is used for coating pills and is commercially available as Opadry-Oy34817 from Colorcon Ltd., Italy.

Other materials may be used as a protective coating for a sensor. The protective coating may be made from any other substance which is hard or thick enough to protect the sensor from damage during insertion, dissolves immediately or soon after insertion and is biocompatible in the intended location of deployment in the body.

A sensor may be coated by any available method for coating objects including, for example, spraying the coating on the sensor, dipping the sensor in a liquid bath, pouring or dripping the coating onto the sensor, painting the coating onto the sensor, etc. Additionally, the coating may cover only the membrane of the sensor or it may cover a larger portion of the sensor or the entire sensor.

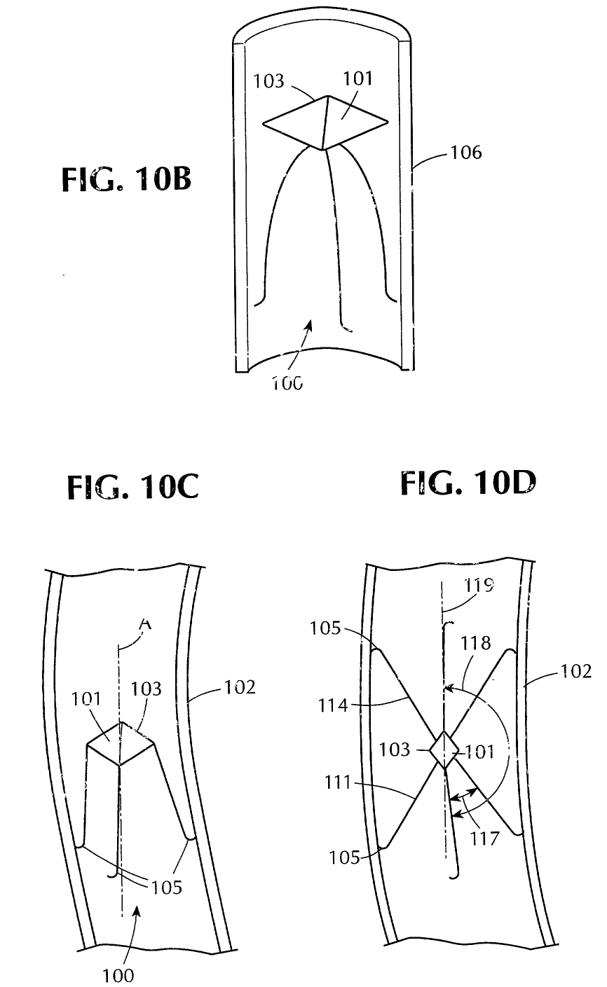

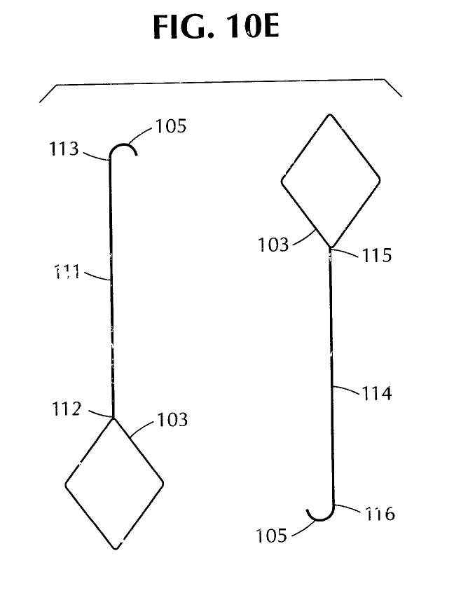

Referring now to FIGS. 10A-10E, an anchor 100 for a sensor 101 is to be positioned at the center of a lumen 102. The anchor in this embodiment that positions the sensor in the center of the lumen has the following advantages over anchors that do not position the sensor in the center of the lumen. First, when the sensor is positioned on the side of the lumen, the sensor may be covered with endothelium. Thus, placing a sensor in the center of the lumen may prevent build-up of tissue in the vessel. Second, for many physical properties, there are large differences between measurements taken in the middle of the lumen as compared to the side of the lumen. For example, blood flow, blood pressure, and other hemo-dynamic properties will vary depending on where the measurement is taken in the lumen. Generally, blood cells congregate in the center of the lumen and typically do not migrate through the vessels close to the wall.

The anchors can be made of various sizes and diameters to accommodate the dimensions of the lumen that the sensor is to be inserted and ultimately implanted. Depending on the embodiment, the anchor may have a sensor support with a diameter, for example but not limited to, less than 5 mm for small blood vessels. The anchor may also be made of a thickness to minimize the impedance of blood flow in the vessel. For larger blood vessels, the anchor may have a sensor support with a diameter, for example but not limited to, about 9 mm. This larger size sensor support may be used where the sensor is subjected to mechanical forces, such as those close to the skin surface. In this case, the anchor provides maximum stability with the larger anchor structure.

FIG. 10A shows an exemplary anchor 100. The anchor 100 may be made of a super elastic material, such as Nickel Titanium, or the like. The super elastic material is strong enough to resists elongation, but resilient to the extent of having spring-like memory when deformed. A sensor frame 103 is provided to hold the sensor 101 in place when the anchor 100 is positioned inside the lumen 102. The sensor frame 103 may be disposed perpendicular to the length of the lumen 102, as shown in 103a, or parallel to the length of the lumen 102, as shown in 103b, though is not limited to those positions. Additionally, the anchor 100 has a plurality of support legs 104. The support legs 104 are attached to the sensor frame 103 at a first end 1041, and have protrusions 105 at a second end 1042, where the protrusions 105 may be attached to the second end 1042, or an extension of the supports legs 104 themselves. The protrusions 105 may be in the shape of a hook, or the like. For example, the protrusion 105 may be, but is not limited to, a structure including a serrated edge, a beveled edge, an adhesive, a magnetic clamp, a mechanical clamp, a suction cup, or any combinations of these structures. The protrusions 105 may be made sharp enough to dig into the tissue however do so with minimum trauma. The sensor frame 103 is disposed at approximately the center of the anchor 100, along its length, although not limited to such a position.

FIG. 10B shows the anchor 100 of FIG. 10A disposed within a catheter 106. The catheter 106 can position the anchor 100 within the lumen 102 with minimal disturbance to flow. The anchor 100 is constrained within the catheter 106 such that the sensor 101 disposed within the sensor frame 103 is not substantially deformed from the center of the anchor 100. The support legs 104 of the anchor 100 are deformed to fit into the catheter 106. At the lumen 102, the anchor 100 is removed from the catheter 106 such that the support legs 104, deformed when disposed in the catheter 106, will return to their non-deformed condition, as shown in FIG. 10C. The protrusions 105 at the second end 1042 of the support legs 104 then attach to the walls of the lumen 102, leaving the sensor 101 positioned at the center of the lumen 102. The center of a lumen 102 is approximately along the length of the lumen 102 in the A direction, equidistant from the walls of the lumen 102.

The positioning of the sensor 101 at the center of the lumen 102 prevents tissue from the walls of the lumen 102 from covering any part of the sensor 101 that might prevent accurate measurement. It is well known in the art that blood cells concentrate in the center of a lumen. Furthermore, it is well known in the art that the fastest blood flow in a lumen is at its center. Therefore, physiological conditions and biological parameters measured by the sensor 101 at the center of the lumen 102 represent a more accurate measurement of the overall conditions and parameters of the lumen 102.

FIG. 10D shows another exemplary anchor 110. Again, the anchor 110 is made of a super-elastic material, such as NiTi, or the like. A sensor frame 103 is provided to hold the sensor 101 in place when it is positioned inside the lumen 102. The sensor frame 103 may be disposed substantially perpendicular to the length of the lumen 102, or substantially parallel to the length of the lumen 102. Additionally, the anchor 110 has a plurality of first support legs 111 and a plurality of second support legs 114.

As shown in FIG. 10E, showing an exemplary first support leg 111 and an exemplary second support leg 114, the first support legs 111 have a first end 112 and a second end 113. The second support legs 114 have a proximal end 115 and a distal end 116. The first support legs 111 are attached to the sensor frame 103 at the first end 112, and have protrusions 105 at the second end 113, where the protrusions 105 may be attached to the second end 113, or an extension of the first support legs 112 themselves, such that the protrusions 105 and first support legs 111 may be integral or made of one piece. The second support legs 114 are attached to the sensor frame 103 at the proximal end 115, and have protrusions 105 at the distal end 116, where the protrusions 105 may be attached to the distal end 114, or an extension of the second support legs 114 themselves, such that the protrusions 105 and the second support legs 114 may be integral or made of one piece. The protrusions 105 may be in the shape of a hook, or the like. The sensor frame 103 is disposed at approximately the center of the anchor 110, along its length.

As described above in regard to FIG. 10B, the anchor 110 is disposed in a catheter 106 and then deployed inside the lumen 102. The protrusions 105 attach to the wall of the lumen 102 when removed from the catheter 106, thereby securing the sensor 101 in the center of the lumen 102. A first angle 117, from the length of at least one first support leg to the central axis 119 in the B direction, and a second angle, 118, from the length of at least one second support leg to the central axis 119 in the B direction are measured. In the exemplary scenario wherein the contour of the lumen 102 is approximately straight, the absolute value of the first angle 117 of at least one first support leg 112 will be approximately between 0 degrees and 90 degrees and the absolute value of the second angle 118 of at least one second support leg 114 will be approximately between 90 degrees and 180 degrees. In the exemplary scenario wherein the contour of the lumen 102 is not approximately straight, the absolute value of the second angle may no longer be approximately between 90 degrees and 180 degrees, depending upon the shape of the contour. Having support legs at a plurality of angles with respect to the central axis 119 provides better stability for the sensor 101.

It should be understood that the above description is only representative of illustrative examples of embodiments and implementations. For the reader's convenience, the above description has focused on a representative sample of possible embodiments, a sample that teaches the principles of the present invention. Other embodiments may result from a different combination of portions of different embodiments. The description has not attempted to exhaustively enumerate all possible variations. The alternate embodiments may not have been presented for a specific portion of the invention, and may result from a different combination of described portions, or that other undescribed alternate embodiments may be available for a portion, is not to be considered a disclaimer of those alternate embodiments. It will be appreciated that many of those undescribed embodiments are within the literal scope of the following claims, and others are equivalent.

What is claimed is:

1. An anchor for a sensor in a bodily lumen, comprising:
at least one support leg having a first end and a second end; and
a surface for receiving the sensor;
a sensor support coupled to the support leg, wherein the sensor support is disposed at the first end of the support leg such that the sensor is positioned about in the center of the lumen.

2. The anchor of claim 1, wherein the support leg further comprises a protrusion about the second end of the support leg to attach to a wall of the lumen.

3. The anchor of claim 2, wherein the protrusion is a hook, a serrated edge, a beveled edge, an adhesive, a magnetic clamp, a mechanical clamp, a suction cup, or any combination thereof.

4. The anchor of claim 1, wherein the anchor is made of an elastomeric material that is compressible, such that the shape of the sensor support is not substantially deformed.

5. The anchor of claim 4, wherein the elastomeric material comprises nickel titanium.

6. The anchor of claim 1, wherein the sensor support is positioned approximately perpendicular to a wall of the lumen.

7. The anchor of claim 1, wherein the anchor is an aneurismal repair device.

8. The anchor of claim 1, wherein the sensor support further comprises at least a first sensor support and a second sensor support displaced apart from one another within the lumen.

9. The anchor of claim 1, wherein a shape of the sensor support is independent of a shape of the anchor.

10. The anchor of claim 1, wherein the sensor is covered with a protective coating.

11. The anchor of claim 1, wherein the sensor support has a diameter of about 9 mm for large lumen and less than 5 mm for smaller lumen such that blood flow through the lumen is minimally impeded.

12. An anchor for a sensor in a bodily lumen, comprising:
a plurality of first support legs, each having a first end and a second end;
a plurality of second support legs, each having a proximal end and a distal end;
a surface for receiving the sensor; and
a sensor support coupled to the first support legs and the second support legs, wherein the sensor support is disposed at the first end of at least one support leg, and the proximal end of at least a second support leg, such that the sensor is positioned about in the center of the lumen.

13. The anchor of claim 12, wherein the first support legs and second support legs further comprise at least one protrusion, wherein at least one protrusion is coupled to the second end of at least one first support leg, and at least one protrusion is coupled to the proximal end of at least one second support leg, to attach to a wall of the lumen.

14. The anchor of claim 12, wherein the sensor support is positioned approximately parallel to a wall of the lumen.

15. The anchor of claim 12, wherein the sensor support is positioned approximately perpendicular to a wall of the lumen.

16. The anchor of claim 12, wherein the anchor is composed of an elastic material.

17. The anchor of claim 16, wherein the elastic material comprises nickel titanium.

18. The anchor of claim 12, wherein the sensor support comprises at least a first sensor support and a second sensor support displaced apart from one another within the lumen.

19. The anchor of claim 18, wherein the anchor the first sensor support is coupled generally adjacent to the first end and the second sensor support is coupled generally adjacent to the second end of the anchor.

20. The anchor of claim 12, wherein the sensor support is formed from a material other than a material of the sensor.

21. The anchor of claim 12, wherein a material of the sensor support is the same as the material of the anchor.

22. A method for attaching a sensor in a bodily lumen, comprising:
inserting into the lumen an anchor of the type having at least one support leg coupled to a sensor support wherein the sensor is disposed in the sensor support; and
securing the anchor within the lumen, such that the sensor is positioned about in the center of the lumen.

23. The method of claim 22, wherein the securing further includes attaching the support leg to a wall of the lumen.

24. The method of claim 22, wherein the insertion of the anchor further includes constraining the anchor in a catheter.

25. The method of claim 24, wherein the insertion of the anchor further includes removing the anchor from the catheter such that at least one protrusion of at least one support leg secures to the wall of the lumen.

26. The method of claim 22, further includes positioning the sensor support approximately parallel to a wall of the lumen.

27. The method of claim 22, further includes positioning the sensor support approximately perendicular to a wall of the lumen.

28. The method of claim 22, further includes placing a second sensor in the lumen by having the sensor support further including at least a first sensor support and a second sensor support displaced apart from one another at a predetermined distance within the lumen.

29. The method of claim 22, wherein the inserting is accomplished during an intervention procedure.

30. A method for attaching a sensor in a bodily lumen, comprising:
inserting into the lumen an anchor of the type having a plurality of first support legs, each having a first end and a second end; a plurality of second support legs, each having a proximal end and a distal end; a surface for receiving the sensor; and a sensor support coupled to the first support legs and the second support legs, wherein the sensor support is disposed at the first end of at least one support leg, and the proximal end of at least a second support leg, such that the sensor is positioned in the center of the lumen; and
securing the anchor within the lumen, such that the sensor is positioned about in the center of the lumen.

31. The method of claim 30, wherein the securing further includes attaching the support leg to a wall of the lumen.

32. The method of claim 30, wherein the insertion of the anchor further includes constraining the anchor in a catheter.

33. The method of claim 30, wherein the insertion of the anchor further includes removing the anchor from the catheter such that at least one protrusion of at least one support leg secures to the wall of the lumen.

34. The method of claim 30, further includes positioning the sensor support approximately parallel to a wall of the lumen.

35. The method of claim 30, further includes positioning the sensor support approximately perpendicular to a wall of the lumen.

36. The method of claim 30, further includes placing a second sensor in the lumen by having the sensor support further including at least a first sensor support and a second sensor support displaced apart from one another at a predetermined distance within the lumen.

37. The method of claim 30, wherein the insertion is accomplished during an intervention procedure.

38. An anchor for a sensor in a bodily lumen, comprising:
at least one support leg having a first end and a second end; and
a sensor support coupled to the support leg, wherein the sensor support is disposed about the first end of the support leg such that the sensor is positioned about in the center of the lumen, and the sensor support is independent of any other fixation device in the lumen.

39. The anchor of claim 38, wherein the support leg further comprises a protrusion about the second end of the support leg to attach to a wall of the lumen.

40. The anchor of claim 38, wherein the protrusion is a hook, a serrated edge, a beveled edge, an adhesive, a magnetic clamp, a mechanical clamp, a suction cup, or any combination thereof.

41. The anchor of claim 38, wherein the anchor is made of an elastomeric material that is compressible, such that the shape of the sensor support is not substantially deformed.

42. The anchor of claim 38, wherein the elastomeric material comprises nickel titanium.

43. The anchor of claim 38, wherein the sensor support is positioned approximately perpendicular to a wall of the lumen.

44. The anchor of claim 38, wherein the anchor is an aneurismal repair device.

45. The anchor of claim 38, wherein the sensor support further comprises at least a first sensor support and a second sensor support displaced apart from one another within the lumen.

46. The anchor of claim 38, wherein a shape of the sensor support is independent of a shape of the anchor.

47. The anchor of claim 38, wherein the sensor is covered with a protective coating.

48. The anchor of claim 38, wherein the sensor support has a diameter of about 9 mm for large lumen and less than 5 mm for smaller lumen such that blood flow through the lumen is minimally impeded.

49. The anchor of claim 38, wherein the sensor support is positioned approximately parallel to a wall of the lumen.

50. A method for attaching a sensor in a bodily lumen, comprising:
inserting into the lumen an anchor of the type having at least one support leg having a first end and a second end, and a sensor support coupled to the support leg, wherein the sensor support is disposed about the first end of the support leg such that the sensor is positioned about in the center of the lumen, and the sensor support is independent of any other fixation device in the lumen; and
securing the anchor within the lumen, such that the sensor is positioned about in the center of the lumen.

51. The method of claim 50, wherein the securing further includes attaching the support leg to a wall of the lumen.

52. The method of claim 50, wherein the insertion of the anchor further includes constraining the anchor in a catheter.

53. The method of claim 50, wherein the insertion of the anchor further includes removing the anchor from the catheter such that at least one protrusion of at least one support leg secures to the wall of the lumen.

54. The method of claim 50, further includes positioning the sensor support approximately parallel to a wall of the lumen.

55. The method of claim 50, further includes positioning the sensor support approximately perpendicular to a wall of the lumen.

56. The method of claim 50, further includes placing a second sensor in the lumen by having the sensor support further including at least a first sensor support and a second sensor support displaced apart from one another at a predetermined distance within the lumen.

57. The method of claim 50, wherein the inserting is accomplished during an intervention procedure.
